(19) United States
(12) Patent Application Publication
(10) Pub. No.: US 2022/0347366 A1
(43) Pub. Date: Nov. 3, 2022

(54) METHOD AND APPARATUS FOR PURIFYING BLOOD

(71) Applicant: B. Braun Avitum AG, Metsungen (DE)

(72) Inventor: Peter Mandry, Dresden (DE)

(21) Appl. No.: 17/602,367

(22) PCT Filed: Apr. 7, 2020
(86) PCT No.: PCT/EP2020/059832

§ 371 Date: Oct. 8, 2021

(30) Foreign Application Priority Data
Apr. 11, 2019 (DE) 10 2019 109 646.4

(51) Int. Cl. A61M 1/34

(52) U.S. Cl. CPC A61M 1/34; A61M 1/3479; A61M 1/3486; A61M 2205/3324

(57) ABSTRACT

A device and method for purifying blood. The method includes the steps of separating the blood plasma from the blood cells, adjusting the pH of the blood plasma to a pH close to an isoelectric point of at least one predetermined protein, treating the blood plasma by ion-exchange chromatography, neutralizing the pH of the blood plasma, and pooling of the blood plasma and blood cells.

### CROSS-REFERENCE TO RELATED APPLICATIONS

This application is the United States national phase entry of International Application No. PCT/EP2020/059832, filed Apr. 7, 2020, and claims the benefit of priority of German Application No. 10 2019 109 646.4, filed Apr. 11, 2019. The contents of International Application No. PCT/EP2020/059832 and German Application No. 10 2019 109 646.4 are incorporated by reference herein in their entireties.

### FIELD

The present invention relates to a method for purifying blood, in particular for removing endotoxins from blood plasma, and to a device for performing such a method. Furthermore, the invention relates to the use of a diethylaminoethyl (DEAE) anion exchanger, in particular a DEAE anion exchanger with tentacle technology, for purifying blood plasma.

### BACKGROUND

According to calculations by the Sepsis Competence Network, about 154,000 people in Germany contract sepsis or blood poisoning every year. Of these, some 56,000 die as a result of the disease. This amounts to some 154 deaths per day, similar to the number of deaths from heart attack and more than those who die of lung cancer. Sepsis therefore ranks third among the causes of death in Germany.

The Sepsis Competence Network provides the following figures for Europe: 550,000 cases and 146,000 deaths per year, mortality rate 26.5%. From a global perspective, too, sepsis is a frequent cause of death according to Sepsis Competence Network data: there are approximately 1,500,000 cases per year, resulting in 500,000 deaths, with a mortality rate of 33.3%.

According to German Sepsis Aid, this means that more than 1,400 people die of severe sepsis every day worldwide. In the USA, the incidence of sepsis has also increased over the decades to about 3 per 1,000 inhabitants per year.

One cause of the severe progress of sepsis and therefore of increased mortality are the bacterial components circulating in a patient's blood after antibiotic therapy, including endotoxins such as lipoteichoic acid (LTA), lipopolysaccharides (LPS), viruses, deoxyribonucleic acid (DNA) and ribonucleic acid (RNA).

Removal of these particles or endotoxins from a patient's blood, for example using suitable adsorber technology, can potentially result in a less severe progression of sepsis, thereby reducing the mortality linked to sepsis.

As a method for removing pathogenic substances such as endotoxins, viruses, deoxyribonucleic acid (DNA) and ribonucleic acid (RNA) from a patient's blood, ion-exchange chromatography using ion exchangers or ion adsorbers is known from the state of the art. In this connection, anion exchangers or anion adsorbers are mainly used for anion-exchange chromatography.

Unfortunately, in addition to the pathogenic substances such as endotoxins, viruses, deoxyribonucleic acid (DNA) and ribonucleic acid (RNA), these anion exchangers also remove vital components from the patient's blood such as coagulation factors.

The situation is further complicated by the fact that coagulation factors do not belong to the acute-phase proteins and are therefore only slowly reproduced in some cases, which is why the unintentional removal of these proteins has particularly serious and long-lasting consequences for patient safety.

This means that there is a risk of internal and external bleeding in the patient when using ion-exchange chromatography, especially anion-exchange chromatography, for the purpose of blood purification due to the undesirable removal of vital blood coagulation factors, which can lead to the patient's death.

One blood purification method known from the state of the art is H.E.L.P. apheresis (heparin-induced extracorporeal LDL precipitation).

In this extracorporeal blood purification method, the first step after blood collection is plasma separation. LDL (low density lipoprotein) cholesterol, fibrinogen and lipoprotein(a) are precipitated from the plasma obtained by adding acetate buffer for acidification and heparin.

Subsequently, the precipitate is removed with a special filter and excess heparin is removed from the treated plasma by means of adsorption. Finally, bicarbonate dialysis is performed to restore the plasma to its initial volume and a physiological pH. The purified plasma is combined with the blood cells and returned to the patient.

H.E.L.P. apheresis is mainly used for the treatment of severe, otherwise therapy-refractory lipometabolic disorders and rheologically caused diseases such as hearing loss.

Furthermore, a method for supporting liver function is known from the state of the art in which the pH of the blood plasma is raised and lowered to reduce the binding strength of protein-bound molecules to albumin. By changing the pH of the blood plasma, the structure or configuration of the albumin changes, which means that the bound molecules are less strongly bound, go into solution more easily and can be removed more easily by means of dialysis as a result.

Anion exchangers (also basic ion exchangers) are ion exchangers in which a cationic group (cation) is covalently bonded to a solid insoluble matrix, while the neutralizing anion (neutral) is only ionically bonded and therefore exchangeable with other anions. Chromatography using anion exchangers (anion-exchange chromatography) is an important tool for the analysis and binding of proteins and nucleic acids or their components, the peptides, amino acids, oligonucleotides and mononucleotides.

Examples of anion exchangers are aminoethyl, diethylaminoethyl (DEAE), quaternary aminoethyl (QAE) and quaternary ammonium groups coupled to cellulose, agarose (agar), dextran (dextrans) or polystyrene. DEAE cellulose is frequently used.

Of the known anion exchangers, diethylaminoethyl (DEAE) ion exchangers with tentacle technology have a very high binding capacity and binding strength, which means they are particularly effective in removing pathogens and endotoxins even in complex solutions such as human plasma. DEAE ion exchangers are among the most effective known endotoxin and virus binders and are used extremely successfully for this purpose in technology, in particular biotechnology, chemical engineering and process technology.

In tentacle technology, special gels are used in chromatography which have thread-like structures, the so-called tentacles. These tentacles carry charges over their entire length which enable them to hold or bind the substances to be separated, e.g. proteins, depending on their charge, much more effectively than normal gels, which have a smaller surface area.

DEAE ion exchangers (also called DEAE adsorbers) remove pathogenic substances such as lipoteichoic acid (LTA) and lipopolysaccharides (LPS), viruses, etc., but they are also very effective in removing important substances or factors of the blood coagulation system from the blood. This disables the blood coagulation factor system and the patient can potentially bleed to death.

In an in vitro experimental set-up, for example, DEAE adsorbers were flowed through with blood plasma and the adsorption of coagulation factors was determined. Factors II, IX and X of the blood coagulation cascade were adsorbed by the DEAE adsorber and largely removed from the blood. The removal of coagulation factors by ion exchangers in the in vitro experiments was also confirmed in a clinical test with healthy subjects. The global coagulation status dropped to a dangerously low level after treatment with the DEAE ion exchanger.

Due to this undesired adsorption of important coagulation factors, a clinical application of DEAE ion exchangers is currently not possible.

The object of the present invention is to mitigate or eliminate the problems known from the state of the art. In particular, the object of the present invention is to provide a clinically applicable method and a device for effective blood purification while at the same time ensuring a high level of patient safety.

### SUMMARY

One aspect of the invention relates to a method for purifying blood, in particular blood plasma, comprising the steps:
separation of the blood plasma from the blood cells,
adjustment of the pH of the blood plasma to a pH close to an isoelectric point of a predetermined protein,
treatment of the blood plasma by means of ion-exchange chromatography,
neutralization of the pH of the blood plasma,
pooling of the blood plasma and the blood cells.

The core idea of the invention is that the charge of proteins, such as coagulation factors, can be selectively and deliberately changed and adjusted by adjusting the pH. The adsorption to the ion exchanger and therefore the removal of proteins such as coagulation factors from the blood is dependent on the charge of the proteins/molecules.

Preferably, the predetermined protein is a coagulation factor, for example Factor I, Factor II, Factor IV, Factor V, Factor VI, Factor VII, Factor VIIa, Factor VIII, Factor IX, Factor X, Factor XI or Factor XII. Preferably, the method for purifying blood is carried out or applied as part of an extracorporeal blood treatment.

Coagulation factors are amphoteric proteins, i.e. proteins that have both positive and negative charges. Which charge predominates depends on the pH of the solution in which the protein/molecule is located. The isoelectric point (IEP or pI) refers to the pH at which the number of positive and negative charges of an amphoteric protein/molecule is exactly equal on statistical average and the entire protein/molecule is therefore electrically neutral.

After the blood has been separated into blood plasma and blood cells, the blood plasma initially has a physiological pH of approx. 7.4. At a physiological pH of approx. pH 7.4, most coagulation factors have a negative charge. If the pH of the plasma is shifted towards lower pH values (i.e. into the acidic range), the negative charge of the coagulation factors becomes lower. When the pH of the blood plasma reaches the isoelectric point of a particular protein/molecule such as a coagulation factor, the charge of that particular protein/molecule is neutral and the protein/molecule or coagulation factor is no longer adsorbed by the ion exchanger and is no longer removed from the blood plasma.

Unlike proteins such as coagulation factors, the endotoxins that play a causal role in sepsis are predominantly non-amphoteric. Non-amphoteric molecules such as LPS, LTA, other endotoxins and viruses generally do not change their charge depending on the pH of the solution in which the endotoxins are found. They are therefore adsorbed by the ion exchanger largely independently of the pH of the surrounding solution.

The core idea of the invention of adjusting the charge of at least one predetermined protein to neutral by adjusting the pH of the solution surrounding this protein to the pH corresponding to the isoelectric point of the predetermined protein is applicable to any chromatography method based on the interaction of electrically charged components (such as ions).

When the pH of the solution surrounding the predetermined protein corresponds to the isoelectric point of the predetermined protein, the electrically neutral predetermined protein is not adsorbed or retained by means of the chromatographic method (e.g. ion-exchange chromatography), which is based on the interaction of electrically charged components (such as ions), while other undesirable blood plasma components such as endotoxins, whose electrical charge is not neutral but predominantly positive or predominantly negative, are retained and effectively removed from the blood plasma by the chromatography method.

It has been shown here that it is sufficient to shift the pH of the blood plasma to the vicinity of the isoelectric point of a predetermined protein in order to prevent unwanted adsorption of this protein to the ion exchanger and thus unwanted removal of this protein from the blood plasma. In other words, it is not necessary for the pH of the blood plasma to correspond precisely to the isoelectric point of a predetermined protein, rather it is sufficient if the pH of the blood plasma is close to or approximates to the isoelectric point of a predetermined protein.

Preferably, the pH to which the blood plasma is adjusted corresponds to the isoelectric point of the predetermined protein to within +1.5/+0.0 pH point, preferably to within +1.5/+0.5 pH point, more preferably to within +1.5/+0.75 pH point, particularly preferably to within +1.5/+1.0 pH point. For example, the isoelectric point may be pH=5.5 and the pH of the blood plasma is adjusted to pH=5.5-pH=7.0 (pH=5.5+1.5/+0.0 pH point), preferably to pH=6.0-pH=7.0 (pH=5.5+1.5/+0.5 pH point), more preferably to pH=6.25-pH=7.0 (pH=5.5+1.5/+0.75 pH point), particularly preferably to pH=6.5-pH=7.0 (pH=5.5+1.5/+1.0 pH point).

According to another aspect of the invention, the pH of the blood plasma can also be adjusted to a pH lower than the isoelectric point of the predetermined protein. For example, the isoelectric point may be pH=5.5 and the pH of the blood plasma is adjusted to any pH lower than pH=5.5. For example, the pH of the blood plasma may be adjusted to lower than pH=5.5. In particular, a pH between pH=5.5 and pH=5.0 is preferred.

According to one aspect of the invention, the predetermined protein is a coagulation factor, in particular Factor II, and/or the pH of the blood plasma is adjusted to a pH which is lower than the isoelectric point of the coagulation factor.

The pH of the blood plasma can be adjusted by means of a direct or indirect supply of protons (H⁺) to the blood plasma, for example in the form of an acidic solution or also as a solid, and/or by means of dialysis of the blood plasma with an acidic buffer, in particular an acetate buffer. Alternatively or additionally, hydroxide ions (OH⁻) can be removed from the blood plasma.

The neutralization of the pH of the blood plasma can be carried out by means of a direct or indirect supply of hydroxide ions (OH⁻) to the blood plasma and/or by means of a dialysis of the blood plasma with a basic buffer, in particular a bicarbonate buffer. Dialysis of the blood plasma with a basic buffer, especially a bicarbonate buffer, has the advantage that excess fluid can be removed from the blood plasma. Alternatively or additionally, protons (H⁺) can also be removed from the blood plasma.

A pH of pH=7.4 or a pH that deviates only insignificantly from pH=7.4 is considered neutral or neutralized. In particular, a pH between pH=7.2 and pH=7.6 is to be understood as neutral or neutralized.

According to one aspect of the invention, the treatment of the blood plasma is performed by anion-exchange chromatography, preferably using a DEAE anion exchanger, in particular a DEAE anion exchanger with tentacle technology.

In principle, however, the present invention is not limited to anion-exchange chromatography, but also includes other types of ion-exchange chromatography, such as cation-exchange chromatography or also various multimodal chromatographies, which preferably include ion-exchange chromatography.

In principle, it would also be conceivable to perform a method according to the invention with cation-exchange chromatography. Cation exchangers are ion exchangers in which an anionic group (anion) is covalently bonded to a solid insoluble matrix, while the neutralizing cation (neutral) is only ionically bonded and therefore exchangeable with other cations.

A method according to the invention may also take into account a number or a plurality of predetermined proteins (for example a plurality of coagulation factors). Here, the different predetermined proteins of the plurality of proteins may each have different isoelectric points.

In this case, the isoelectric points of all proteins or the majority of proteins are taken into account when adjusting the pH of the blood plasma before ion-exchange chromatography. Generally speaking, the pH of the blood plasma is adjusted to a pH at which all proteins of the majority of predetermined proteins have a desired charge (positive, neutral, negative).

For example, the pH of the blood plasma may be adjusted to an average of the pH values corresponding to the isoelectric points of the proteins of the plurality of predetermined proteins. Alternatively, the pH of the blood plasma may be adjusted to the lowest or the highest of the pH values corresponding to the isoelectric points of the proteins of the plurality of predetermined proteins.

Another aspect of the present invention relates to a programme product which, when read by a device, causes a device to perform a method according to the invention. By means of such a programme product, existing devices can be retrofitted to perform a method according to the invention.

Another aspect of the invention relates to a blood treatment machine, in particular an apheresis machine, which is designed or configured to perform a method according to the invention.

Such a blood treatment machine comprises, for example:
a plasma filter for separating blood into blood plasma and blood cells,
a feed device/mixing pump fluidically connected downstream of the plasma filter for adjusting the pH of the blood plasma,
an ion exchanger downstream of the feed device/mixing pump, which is preferably an anion exchanger, and
a dialyzer fluidically connected downstream of the ion exchanger, by means of which dialysis can be carried out using acidic or basic dialysis fluid.

Here, the ion exchanger is preferably an anion exchanger, for example a DEAE anion exchanger, in particular a DEAE anion exchanger with tentacle technology. The ion exchanger can also be configured for multimodal chromatography, for example with additional hydrophobic interactions.

The pH of the blood plasma can be adjusted by means of the feeding device/mixing pump, which directly or indirectly feeds protons (H⁺) to the blood plasma, for example in the form of an acidic solution or also as a solid.

Alternatively or additionally, the pH can also be adjusted by means of dialysis of the blood plasma with an acidic buffer, in particular an acetate buffer. A dialyzer fluidically connected upstream of the ion exchanger can be provided for this purpose, by means of which dialysis can be carried out using acidic dialysis fluid (and in principle also basic dialysis fluid).

The neutralization of the pH of the blood plasma can be carried out by means of the feeding device/mixing pump, which directly or indirectly feeds hydroxide ions (OH⁻) to the blood plasma, for example in the form of a basic solution or also as a solid.

Alternatively or additionally, the neutralization of the pH of the blood plasma can also be carried out by means of dialysis of the blood plasma with a basic buffer, in particular a bicarbonate buffer. Dialysis of the blood plasma with a basic buffer, especially a bicarbonate buffer, has the advantage that excess fluid can be removed from the blood plasma.

Since the neutralization of the pH of the blood plasma takes place after the blood plasma has flowed through the ion exchanger, a dialyzer fluidically downstream of the ion exchanger is preferably provided for this purpose, by means of which dialysis can be carried out using basic dialysis fluid (and in principle also acidic dialysis fluid).

Another aspect of the invention relates to the use of a DEAE anion exchanger, in particular a DEAE anion exchanger with tentacle technology, for the purpose of extracorporeal blood treatment.

In other words, the present invention enables anion exchangers, in particular DEAE anion exchangers, in particular DEAE anion exchangers with tentacle technology, to be used for extracorporeal blood treatment/for extracorporeal preparation/purification of blood or blood plasma.

In this way, blood plasma can be effectively and selectively purified from endotoxins without unintentionally removing important components of the blood plasma, such as coagulation factors.

According to this aspect, the method according to the invention can be used, for example, in blood banks for treating donor blood. A method according to the invention can also be used as part of an extracorporeal blood treatment.

In other words, according to one embodiment, the invention relates to a method comprising the steps:
separation of the blood plasma from the blood cells,
enrichment of the blood plasma with H⁺ ions (protons) until the pH of the blood plasma approaches the isoelectric point of Coagulation Factor II (pH of the blood plasma is lower than pH=5.5),
use of anion-exchange chromatography/an anion exchanger to remove pathogens from the blood plasma,
neutralization of the pH of the blood plasma to approx. pH 7.4,
pooling of the blood plasma and blood cells.

Neutralization of the blood plasma can be carried out by any method, for example by adding a base, OH⁻ ions, by removing the H⁺ ions or by means of a basic buffer, such as a bicarbonate buffer.

Lowering the pH of the blood plasma can be achieved by adding H⁺ ions in various forms, for example by adding an acid such as HCl or a buffer with an acidic pH such as an acetate buffer.

Neutralization of the pH of the blood plasma after it has passed through the ion exchanger can be achieved by adding a base, such as NaOH, or by dialysis with a basic buffer, such as a bicarbonate buffer.

For example, after separating the blood plasma from the blood cells, an acetate buffer with a pH of approx. 4 is added to the plasma, thus lowering the pH of the plasma to approx. pH 5. The blood plasma then flows through the ion exchanger.

After the ion exchanger, the blood plasma flows through a dialyzer. Bicarbonate buffer preferably flows through this on the dialysate side. In other words, a bicarbonate buffer is used as the dialysis fluid. This restores the neutral pH or physiological pH of the blood plasma and removes any additional fluid that may have been introduced into the blood plasma by ion-exchange chromatography, for example.

In this purified state, the blood plasma has its physiological composition (including a physiological pH and blood coagulation factors present in the blood plasma) and is effectively cleansed of any endotoxins previously present in the blood plasma, such as LPS, LTA, viruses, etc.

### BRIEF DESCRIPTION OF THE DRAWING FIGURES

FIG. 1 shows an example of the adsorption of various coagulation factors on a DEAE anion exchanger at a physiological pH of the blood plasma.

FIG. 2 shows an example of the adsorption of various coagulation factors on a DEAE anion exchanger at a blood plasma pH of pH=5.1.

FIGS. 3a and 3b illustrate the pH-dependent adsorption of various coagulation factors on a DEAE anion exchanger.

FIG. 4 shows an example of a device according to the invention.

### DETAILED DESCRIPTION

Examples of embodiments of the present invention are described below with reference to the accompanying figures.

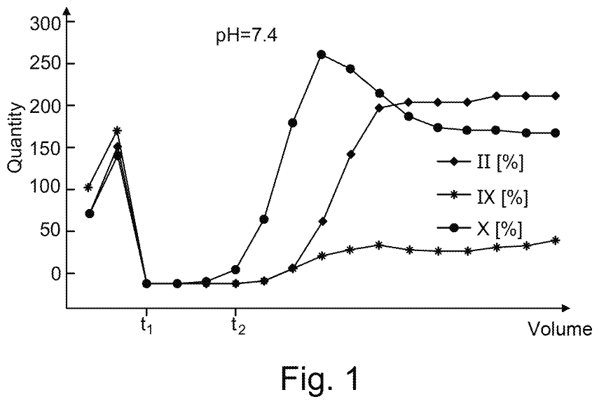

FIG. 1 shows an example of the adsorption of various coagulation factors on a DEAE anion exchanger at a physiological pH of the blood plasma. In an in vitro experimental set-up, DEAE anion exchangers were flowed through with blood plasma with a physiological pH of pH=7.4 and the adsorption of coagulation factors was determined.

Factors II, IX and X of the blood coagulation cascade were effectively adsorbed by the DEAE anion exchanger and largely removed from the blood. At a time t1, the measurable amount of Coagulation Factors II, IX and X in the blood plasma is zero. The global coagulation status therefore dropped to a dangerously low level after treatment with the DEAE anion exchanger.

The amount of Coagulation Factor IX remained permanently low, whereas Coagulation Factors II and X were no longer adsorbed after a plasma volume (approx. 3 L, after a time t2 in FIG. 1) was passed through the DEAE anion exchanger. Therefore, the amounts of Coagulation Factors II and X increase after time t2 in FIG. 1.

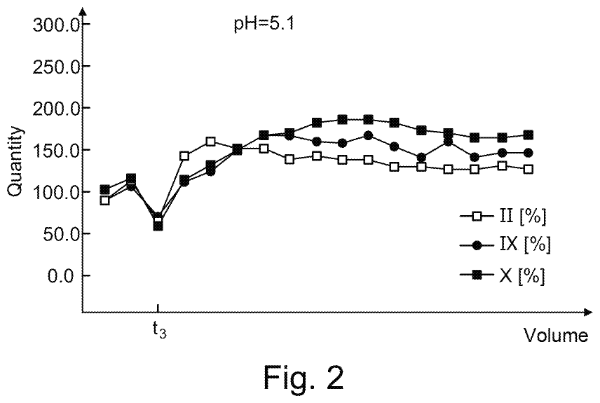

FIG. 2 shows an example of the adsorption of various coagulation factors on a DEAE anion exchanger at a blood plasma pH of pH=5.1.

The amounts of Coagulation Factors II, IX and X remain stable and there is no adsorption to the DEAE anion exchanger. The dip of the curves at time t3 in FIG. 2 is an artefact and is due to a dilution effect of rinsing solution present in the system.

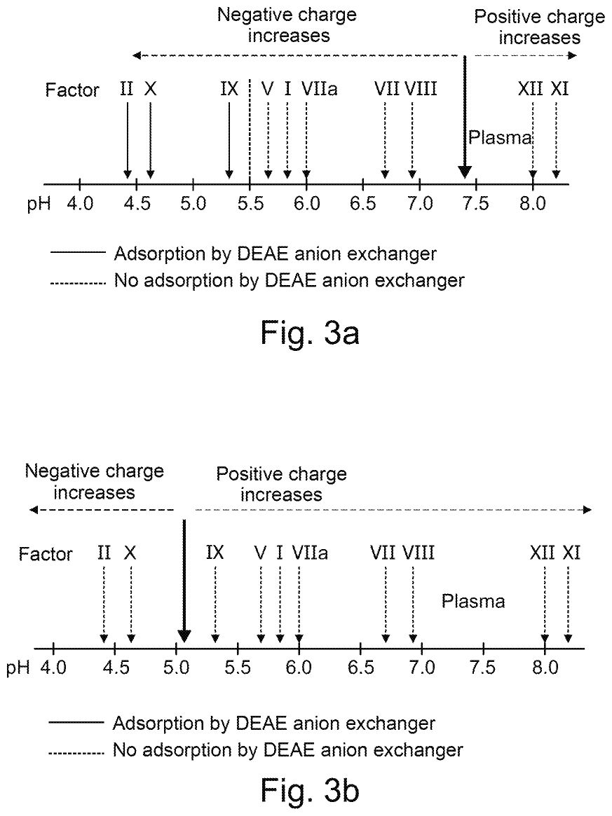

FIGS. 3a-b illustrate the pH-dependent adsorption of the coagulation factors Factor I, Factor II, Factor IV, Factor V, Factor VI, Factor VII, Factor VIIa, Factor VIII, Factor IX, Factor X, Factor XI and Factor XII to a DEAE anion exchanger. The position of the vertical arrows on the pH scale shows the isoelectric point of the respective coagulation factors. FIG. 3a shows the adsorption of the coagulation factors when the blood plasma has a pH of approx. pH 7.4. FIG. 3b shows the adsorption of the coagulation factors when the blood plasma has a pH of approx. pH 5.1.

As shown in FIG. 3a, at a blood plasma pH of approx. pH 7.4, Factors II, IX and X are adsorbed on the DEAE anion exchanger, while Factors V, I, VIIa, VII, VIII, XII and XI are not adsorbed. When the pH shifts from the neutral pH of pH 7.4 into the acidic range (lower pH), the negative charge of the proteins/molecules increases. When the pH shifts from the neutral pH of pH 7.4 into the basic range (higher pH), the positive charge of the proteins/molecules increases.

As shown in FIG. 3b, at a blood plasma pH of approx. pH 5.1, there is no adsorption of Factors II, IX, X, V, I, VIIa, VII, VIII, XII and XI on the DEAE anion exchanger. The pH of approx. 5.1 is close to the isoelectric point of Factor II, which is pH=4.4 and is 1.1 higher than the isoelectric point of Factor II.

When the pH shifts from the acidic pH of pH 5.1 further into the acidic range (lower pH), the negative charge of the proteins/molecules increases. When the pH shifts from the acidic pH of pH 5.1 to the basic range (higher pH), the positive charge of the proteins/molecules increases.

In both the case shown in FIG. 3a and in the case shown in FIG. 3b, non-amphoteric pathogenic substances such as endotoxins, viruses, deoxyribonucleic acid (DNA) and ribonucleic acid (RNA) are bound by the DEAE anion exchanger with consistent effectiveness and therefore removed from the blood plasma.

By adjusting the pH of the blood plasma prior to ion-exchange chromatography, the adsorption or binding behaviour of amphoteric proteins/molecules such as coagulation factors can therefore be selectively influenced in such a way that these proteins/molecules are not adsorbed or removed from the blood plasma, while non-amphoteric proteins/molecules such as pathogens and endotoxins continue to be effectively adsorbed and therefore removed from the blood plasma.

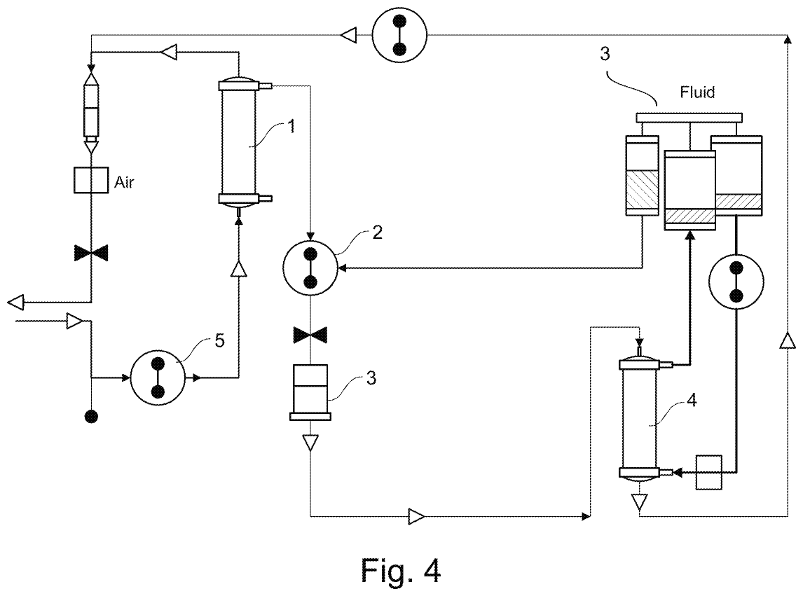

FIG. 4 schematically shows the structure of a blood treatment machine according to the invention, which is designed/configured to perform a method according to the invention. The blood treatment machine is preferably an apheresis machine.

The blood machine is connected or connectable to a patient via a blood inflow line and a blood outflow line. The blood from the blood inflow line is conveyed by a first feed pump 5 and then flows further into a plasma filter 1 having two outlets, in which the blood is separated into blood plasma and blood cells, whereby the blood plasma flows out from one outlet of the plasma filter 1 and the blood cells flows out from another outlet of the plasma filter 1.

The blood plasma separated by the plasma filter 1 is further mixed in a feed device/mixing pump 2 with an acetate buffer having a pH of pH 4 provided by a fluid supply/source 6 to lower the pH of the blood plasma to approx. 5.

The blood plasma treated in this way is channeled by the feed device/mixing pump 2 to a valve/shut-off device for controlling the flow. From there it flows further into an ion exchanger 3, which in this embodiment is an anion exchanger, preferably an anion exchanger with tentacle technology.

Ion exchange with charges of the molecules takes place in the ion exchanger 3. If the pH of the blood plasma is close to the isoelectric point, the amphoteric coagulation factor is no longer adsorbed by ion exchanger 3. Non-amphoteric molecules such as LPS, LTA and viruses are further adsorbed in ion exchanger 3 regardless of the pH of the blood plasma.

After the ion exchanger 3, the blood plasma flows through a blood side of a dialyzer 4, in which the dialysate side of the dialyzer 4 is flowed through with a bicarbonate buffer provided from a further fluid supply/source 6 as dialysing fluid to restore a neutral physiological pH of the blood plasma, whereby a second feed pump 5 is arranged between the fluid supply/source 6 with bicarbonate buffer and an inlet of the dialyzer 4 to feed the bicarbonate buffer.

The purified blood plasma, which again has a physiological pH, is further conveyed by a third feed pump 5 downstream of the dialyzer 4 and then reunited with the blood cells from the plasma filter 1.

The treated blood then continues to flow through an air trap with an air detector, in which air bubbles are detected and removed, and flows to another downstream valve/shut-off device to control the flow which is connected to the blood discharge line. From there, the treated blood is returned to the patient.

In summary, the blood treatment machine comprises a plasma filter 1 for separating blood into blood plasma and blood cells, a feed device/mixing pump 2 fluidically connected downstream of the plasma filter 1 for adjusting the pH of the blood plasma, an ion exchanger 3, which is preferably an anion exchanger, connected downstream of the feed device/mixing pump 2, and a dialyzer 4, which is fluidically connected downstream of the ion exchanger 3 and by means of which dialysis can be carried out using acidic or basic dialysis fluid.

After the blood has been separated into blood plasma and blood cells in the plasma filter 1, the blood plasma is mixed with an acetate buffer with a pH of pH 4 provided by a fluid supply/source 6 by means of the feed device/mixing pump 2 to lower the pH of the blood plasma to approx. pH 5.

The blood plasma then flows through a valve/shut-off device into the ion exchanger 3, which in this embodiment is an anion exchanger, preferably an anion exchanger with tentacle technology.

After the ion exchanger 3, the blood plasma flows through the dialyzer 4. This is flowed through on the dialysate side with a bicarbonate buffer provided as dialysis fluid by another fluid supply/source 6 and conveyed by a second feed pump 5. In this way, the dialyzer 4 restores a neutral physiological pH of the blood plasma and removes additional fluid from the blood plasma.

The purified blood plasma is then reunited with the blood cells from the plasma filter 1 by a third feed pump 5. The combined and purified blood flows through an air trap with an air detector and another downstream valve/shut-off device and finally from the blood treatment machine back to the patient.

1. A method for purifying blood comprising the steps of:
separating blood plasma from blood cells;
adjusting a pH of the blood plasma to a pH close to an isoelectric point of at least one predetermined protein;
treating the blood plasma by ion-exchange chromatography;
neutralizing the pH of the blood plasma; and
pooling the blood plasma and the blood cells,
wherein the pH to which the pH of the blood plasma is adjusted corresponds to the isoelectric point of the predetermined protein to within +1.5/+0.0 pH point.

2. (canceled)

3. (canceled)

4. The method according to claim 1, wherein the predetermined protein is a coagulation factor.

5. The method according to claim 1, wherein the pH of the blood plasma is adjusted to a pH which is lower than pH=5.5.

6. The method according to claim 1, wherein the adjustment of the pH of the blood plasma is carried out by supplying protons to the blood plasma and/or dialysis of the blood plasma with an acidic buffer.

7. The method according to claim 1, wherein neutralization of the pH of the blood plasma is carried out by supplying hydroxide ions to the blood plasma and/or dialysis of the blood plasma with a basic buffer.

8. The method according to claim 1, wherein treatment of the blood plasma is carried out by means of anion-exchange chromatography.

9. (canceled)

10. A blood treatment machine configured to perform the method according to claim 1, the blood treatment machine comprising:
a plasma filter for separating blood into blood plasma and blood cells;
a feed device/mixing pump for adjusting a pH of the blood plasma; and
an ion exchanger.

11. The blood treatment machine according to claim 10, further comprising a dialyzer, wherein:
the feed device/mixing pump is fluidically connected downstream of the plasma filter,
the ion exchanger is connected downstream of the feed device/mixing pump, and
the dialyzer is configured to carry out dialysis using acidic or basic dialyzing fluid.

12. The blood treatment machine according to claim 10, wherein the ion exchanger is a DEAE anion exchanger.

13. A method of purifying blood plasma comprising the step of using a DEAE anion exchanger to purify the blood plasma.

14. The method according to claim 1, wherein the pH of the blood plasma is adjusted to a pH in a range between pH=5.0 and pH=5.5.
